(19) United States
(12) Plant Patent Application Publication
(10) Pub. No.: US 2003/0014799 P1
(43) Pub. Date: Jan. 16, 2003

(54) Novel rose scented geranium pelargonium graveolenes plant 'Safal'

(75) Inventors: Sushil Kumar, Uttar Pradesh (IN); Ritika Gupta, Uttar Pradesh (IN); Kakaraparthi Pandu Sastry, Uttra Pradesh (IN); Suchitra Banerjee, Uttra Pradesh (IN); Gopal Rao Mallavarapu, Uttar Pradesh (IN); Srinivas Iyer Ramesh, Uttar Pradesh (IN); Ajit Kumar Shasany, Uttar Pradesh (IN); Mahendra Pandurang Darokar, Uttar Pradesh (IN); Suman Preet Singh Khanuja, Uttar Pradesh (IN)

Correspondence Address: ROTHWELL, FIGG, ERNST & MANBECK, P.C., 1425 K STREET, N.W., SUITE 800, WASHINGTON, DC, 20005, US

(73) Assignee: Council of Scientific & Industrial Research, New Delhi (IN)

(21) Appl. No.: 09/799,880

(22) Filed: Mar. 7, 2001

(51) Int. Cl. A01H 5/00

(52) U.S. Cl. PLT/324

(57) ABSTRACT

The invention relates to a new, distinct and unique plant of rose scented geranium Pelargonium graveolens ‘Safal’ derived from the spontaneous half-sib seed progeny of largely sterile populations of the cultivar accession ‘Bipuli’, possessing the following combination of characteristics namely demonstrated vigour in the essential oil yield related traits in great measure and out yielded all the other accessions; the essential oil of the plant has 89% rhodinol content in which citronellol to geraniol ratio is approximately 1:1 and the contents of isomenthone, menthone, 10-epi-γ-eudesmol, 6,9-guaiadiene, decanoic acid and isodecanoic acid were relatively lower than in the oils of accessions ‘Bipuli’ and/or ‘Hemanti’, this plant can be propagated vegetatively through stem cuttings and suitable for commercial cultivation in large scale.

### FIELD OF INVENTION

The present invention is related to the development of a novel high essential oil producing plant obtained through a unique method of progeny screening from the seeds obtained from the plant cultivar ‘Bipuli’ of Pelargonium graveolens, from which a novel, unique and commercially viable plant of rose scented geranium Pelargonium graveolens with high quantity and quality of essential oil yield was screened out. Further, the invention relates to the development of a high essential oil yielding hybrid named as ‘Safal’ through a planned selection and analysis of the ‘Bupuli’ seedling progenies. The essential oil of the plant is rich in rhodinol (89%) which includes the constituents geraniol, citronellyl, geranyl acetate, citronellyl acetate, geranyl formate, citronellyl formate, phenyl ethyl alcohols, cis and trans rose oxides, linalool and likewise. The hybrid of the invention can be planted and maintained for commercial cultivation through vegetative propagation using the stem cuttings.

### BACKGROUND OFTHE INVENTION

The members of the genus, Pelargonium commonly known as geraniums, are common ornamental plants. Some of the species such as P. graveolens are used as the source of rose scented geranium essential oil of considerable commercial importance. Geranium oil is one of the expensive essential oils used in perfumery, cosmetics and soap industries. On account of its antimicrobial and pesticidal activities, the use of geranium essential oil for technical applications is expanding. There is need to expand the geranium cultivation in diverse geographical areas with suitable agroclimates to meet the increasing industrial demand for the geranium essential oil. Traditionally, the geranium cultivation is confined to areas having semi-temperate to temperate climates where geranium is planted as perennial crop, maintained in the field for 4-5 years. The shoot portion of the perennial plantations of geranium is harvested 1 to 3 times each year to distill the oil. The spread of geranium cultivation has been narrow because the prevalent cultivars of Pelargonium graveolens are highly susceptible to water logging in soil, white ants and several bacterial and fungal diseases. Also there is paucity of genetic resources in Pelargonium species cultivated for essential oils. The available varieties suffer from high degree of male and female sterility. Breeding programmes based on cross hybridization have been scarce among essential oil yielding geraniums. So it is important to generate more genotypes with varied characters which can yield high quantity and quality of essential oil.

### OBJECTS OF THE INVENTION

The main object of the present invention is to develop a new and distinct plant of Pelargonium graveolens ‘Safal’, through progeny screening from the seeds obtained from the plant cultivar ‘Bipuli’ of Pelargonium graveolens, said plant capable of producing higher quantity and quality of essential oil.

Another object of the present invention is to develop a new plant, which produces less of isomenthone, menthone and other sesqui-terpenes but rich in total rhodinol with a citroniol:geraniol ratio of 1:1.

### DESCRIPTION OF THE IVENTION

Accordingly, in the present invention we have developed a novel, distinct, unique, and high essential oil yielding plant of geranium (Pelargonium graveolens) ‘Safal’, through progeny screening from the seeds obtained from the plant cultivar ‘Bipuli’ of Pelargonlum graveolens, possessing the following combination of characters:

a. the plant is a hybrid between cultivar ‘Bipuli’ and cultivar ‘Hemanti’ as the pollen donor as indicated by the co-dominance RAPD pattern obtained by the random primer 5′AACGTACGCG3′

b. possessing very large, hairy, soft, green (137C) leaves, dark pink (78D) petals in the flowers,

c. possessing vigorous and rapid vegetative growth with higher plant height of up to 82±6 cm, higher canopy size of up to 1.42±0.4 m², higher herb yield of up to 11.1±0.9 kg per plant, higher leaf area of up to 101±16 cm², higher oil content up to 0.35 to 0.40% and higher oil yield of up to 41 g/plant,

d. producing an essential oil with the following composition; Citronellol 30.6±3.0, Geraniol 28.7±6.0, Isomenthone 8.4±0.5, Linalool 4.7±0.1, Cis rose oxide 0.4±0.1, trans rose oxide 0.2±0.1, Menthone 0.2±0.1, Citronellyl formate 6.6±0.5, Geranyl formate 2.9±0.2, 10-epi-γ-eudesmol 5.4±0.3, 6,9-guaiadiene 0.1±0.1, Decanoic acid 0.1±0.1, Phenyl ethyl tiglate 0.8±0.1, which may not be construed to be limited to these values,

e. with distinct molecular profile by random amplified polymorphic DNA (RAPD) using 10 random primers (AAATCGGAGC, GTCCTACTCG, TGCGCGATCG, AACGTACGCG, CGGGATCCGC, GCGAATTCCG, CCCTGCAGGC, CCAAGCTTGC, AAGATAGCGG, GGATCTGAAC) distinguishing the plant from the other existing varieties,

f. producing highest herbage, oil yield per plant as compared to any other existing varieties, and

g. possessing the following botanical details

Stem shape: ROUNDED

Stem habit: RAMIFIED

Number of nodes

(i) Primary nodes.—4.

(ii) Secondary nodes.—25 to 30.

(iii) Tertiary nodes.—7.

Average length of primary internode: 3.0 cm

Leaf apex shape: MUCRONATE (rounded), CUSPIDATE

Leaf shape: PALMATELY LOBED, CORDATE

Leaf lamina base shape: HASTATE

Petiole shape: LONG, HAIRY

Color of upper leaf surface: YELLOW GREEN (147A)

Color of lower leaf surface YELLOW GREEN (146A)

Number of trichomes: 3.5/mm²

Trichome ratio: 2:1 (lower leaf/upper leaf)

Time for flowering: FEBRUARY END

Lastingness of bloom: 3 MONTHS

Flower shape: TUBULAR

Pedicel length: 2.5 mm

Pedicel color: YELLOW GREEN

Calyx diameter: 5 mm

Calyx color: YELLOW GREEN (146A)

Corolla: FINE, POLY PETALOUS, TWO POSTERIOR PETALS LARGER WITH REDDISH PURPLE MARKINGS

Corolla color: RED PURPLE (74B)

Pubescence of corolla: ABSENT

Number of anthers: 10

Color designation: YELLOW

Stigma:

Ovaries: ONE SUPERIOR, PENTA CARPELLARY SYNCARPOUS OVARY

Color of stigma: MAGENTA (RED PURPLE)(78B)

Color of ovaries: WHITE

The plant ‘Safal’ can yield more essential oil with less of isomenthone, menthone and other sesqui-terpenes but rich in total rhodinol with a citroniol: geraniol ratio of 1:1, than the prevailing cultivars now grown in India.

While observing large populations of the ‘Bipuli’, ‘Hemanti’ and ‘Kunti’ cultivars of rose scented geranium P. graveolens growing at the Kodaikanal field station Tamil Nadu, India of this Institute, in the temperate climate of Southern hills, formation of fruits bearing seeds at low frequency was observed in the populations of the ‘Bipuli’ cultivar of P. graveolens. It was realized that the seeds obtained may be the product of rare self fertilization within ‘Bipuli’ cultivar or cross pollination of ‘Bipuli’ gynoecia by fertile pollen grains formed on ‘Hemanti’ or ‘Kunti’ cultivar. The present work was carried out to reveal new genetic variation if any present, among the plants that could be raised from the spontaneous seeds borne on the plants of the ‘Bipuli’ cultivar. Comparison of the plants produced from seeds, collected from ‘Bipuli’ cultivar plants growing amongst those of ‘Hemanti’ and ‘Kunti’ cultivar, has shown that the ‘Bipuli’ seed progeny plants indeed differed from the plants of the three cultivars of P. graveolens. Out of all progenies, one (BSP-4) named as ‘Safal’ was found to be high yielding with essential oil containing the oil constituents in desirable proportions, novel and was thus the plant of this invention.

All the color grouping given in the description are made as per The International Royal Horticultural Society Index.

### BRIEF DESCRIPTION OF THE ACCOMPANYING DRAWINGS

FIG. 1 shows Co-dominance pattern of RAPD profile for the plant ‘Safal’ with Primer 5′AACGTACGCG3′.

FIG. 2 shows Unique RAPD profile of ‘Safal’.

FIG. 3 is a Photograph of plant ‘Safal’.

### BREEDING HISTORY

The plants of Bipuli’, ‘Kunti’ and ‘Hemanti’ varieties of P. graveolens were planted in beds of 20 m² size arranged randomly in a field at the experimental farm of Central Institute of Medicinal and Aromatic Plants (CIMAP), located at Kodaikanal at 101° N latitude, 78° E longitude and 1800 m above sea level in the state of Tamil Nadu, India, having temperate agroclimatic features. The distinguishing characteristics of the three accessions (varieties) are given in the Table 1. Table 2 gives the detailed botanical description of the newly developed plant ‘Safal’. The flowers of all the three varieties were generally sterile, however a small number of fruits bearing seed were found to have come up in the year 1997 on the plants of the ‘Bipuli’ cultivar. The seeds were separated from the fruits, soaked in 100 ppm GA₃ solution in water for 24 hours and sown in earthen trays containing 1:1 ratio of soil and farmyard manure in a glasshouse at Lucknow, India (26.5° N 80.5° E and of 120 m altitude, in subtropical north Indian plains), India. Altogether four seedlings were recovered from three separate sowings of 10 seeds each. The seedlings were transplanted individually into pots carrying soil and sand mixture and were continued to be maintained in the glasshouse under 16 hour light and 8 hour dark conditions. Cuttings from these were used to multiply the new accessions called BSP-1 to BSP-4 (BSP-Bipuli seed progeny).

TABLE 1
Distinguishing features of the cultivars Bipuli, Hemanti and Kunti compared to the Bipuli seed progenies.

| SI. | | Cultivar accessions | | | Bipuli seed progeny accessions | | | |
|---|---|---|---|---|---|---|---|---|
| No. | Character | cv. Bipuli | cv. Hemanti | cv. Kunti | BSP-1 | BSP-2 | BSP-3 | BSP-4 |
| 1. | Habit | Semi-erect | Prostrate | Erect | Erect | Erect | Erect | Semi-erect |
| 2. | Canopy | Spread has 100 | Spread has 80 | Spread has 60 | Spread has 42 | Spread has 40 | Spread has 68 | Spread has 140 |
| | | cm diameter and | cm diameter | cm diameter | cm diameter and | cm diameter and | cm diameter and | cm diameter and |
| | | 50 cm height | and 55 cm | and 40 cm | 48 cm height | 45 cm height | 52 cm height | 80 cm height |
| | | | height | height | | | | |
| 3. | Stem | Moderately | Highly hairy, | Poorly haired, | Poorly haired, | Poorly haired, | Moderately | Moderately |
| | | hairy, strong, | weak, 2-3 | sturdy usually | sturdy, 3 | sturdy, 3-4 | hairy, weak, 3 | hairy, sturdy, |
| | | 4-5 primary | primary | 1-2 primary | primary | primary | primary | 5 primary |
| | | branches which | branches which | branches which | branches which | branches which | branches which | branches which |
| | | give out 20-25 | are highly | bear 15-20 | give out 15-20 | give out 10-50 | give out 15-20 | are highly |
| | | secondary | branched into | secondary | secondary and 3 | secondary | secondary | branched into |
| | | branches and | 25-30 | branches and | tertiary | branches and | branches and | 35-40 secondary |
| | | 5-7 tertiary | secondary | 3-5 tertiary | branches | 3-4 tertiary | 3-5 tertiary | branches and |
| | | branches | branches, 5 | branches | | branches | branches | 5-7 tertiary |
| | | | tertiary | | | | | branches |
| | | | branches | | | | | |
| 4. | Leaf | Medium size, | Long, thin, | Small, stout, | Short, stout, | Very short, | Long, thin | Very long, soft |
| | petiole | thin rough dark | soft, light | rough, | rough, faint, | stout, rough, | soft, without | pink at the |
| | | pink at base | pink at base | anthocyanin | anthocyanin | light pink at | any | base |
| | | | | pigment absent | present at the | the base | pigmentation at | |
| | | | | | base | | base | |
| 5. | Leaf lamina | Small (5 cm | Large (8 cm | Very large (9 | Large (9 cm | Large (9 cm | Medium (8 cm | Very large (10 |
| | | long and 6 cm | long, 12 cm | cm long and 14 | long and 12 cm | long and 12 cm | long and 10 cm | cm long and 13 |
| | | wide), lamina | wide), lamina | cm wide), | wide), lamina | wide), lamina | wide) lamina | cm wide), |
| | | has about 75 | has about 60 | lamina has 45 | has 75 lobes, | has 80 lobes, | has 60 lobes, | lamina has 60 |
| | | lobes, hairy, | lobes, very | lobes, | leathery, | leathery, | soft, yellowish | lobes, hairy, |
| | | yellowish green | hairy, dark | leathery, | yellowish green | yellowish green | green 144A^{a} | soft, green |
| | | 146A^{a} | green 137B^{b} | yellowish green | 146A^{a} | 146A^{a} | | 137C^{b} |
| | | | | 146A^{a} | | | | |
| 6. | Leaf | Medium | Long (800 μm), | Short | Very short (250 | Very short (200 | Short (300 μm), | Medium (350 |
| | trichomes | (400 μm), thin | thin | (300 μm), stout | μm), stout | μm), stout | thin | μm), thin |
| 7. | Flower | Medium size | Big size | Small size (1.0 | Medium size 1.3 | Medium size | Small size (1 | Medium size |
| | | ((1.5 cm), dark | (1.5-2.0 cm), | cm), petals, | cm), pink | (1.3 cm), | cm), light pink | (1.3 cm) dark |
| | | pink petals | pink petals | light pink | petals (74B^{e}), | purple petals | petals (74D^{e}), | pink petals |
| | | (78C^{c}), yellow | (80B^{d}), anthers | (74D^{e}), pink | pink fertile, | (77C^{c}), pink | yellow anthers, | (78D^{c}), yellow |
| | | fertile | incompletely | fertile | anthers, seeds | fertile | seeds not | anthers seeds |
| | | anthers, seeds | developed in | anthers, seeds | formed | anthers, seeds | formed | occasionally |
| | | formed | the form of | formed | | formed | | formed |
| | | occasionally | staminodes | | | | | |
| 8. | Shoot | 0.20-0.25% | 0.10-0.15% | 0.25-0.30% | 0.31-0.35% | 0.35-0.40% | 0.25-0.30% | 0.35-0.40% |
| | essential | | | | | | | |
| | oil content | | | | | | | |
| 9. | Physical | Bright yellow | Pale yellow | Greenish | Greenish yellow | Greenish yellow | Bright yellow | Pale yellow |
| | appearance | | greenish yellow | yellow | | | | |
| | of oil | | | | | | | |
| 10. | Citronel- | 1:1 like in | 3-4:1 like that | 1:5 | 1:3 | 1:2.5 | 1.5:1 | 1:1 |
| | lol:geraniol | Bourbon type | in commercial | | | | | |
| | ratio | commercial oil | oil of Chinese | | | | | |
| | | of Reunion | origin | | | | | |
| | | Island origin | | | | | | |

^{a}yellow green group;
^{b}green group;
^{c}purple group;
^{d}purple violet group;
^{e}red purple group;
*(all color groupings were made as per the International Royal Horticultural Society Index)

TABLE 2
Detailed Botanical Description of the plant
‘Safal’:

| | | |
|---|---|---|
| 1. | Stem shape: | ROUNDED |
| 2. | Stem habit: | RAMIFlED |
| 3. | Number of nodes | |
| | (i) Primary Nodes: | 4 |
| | (ii) Secondary Nodes: | 25 to 30 |
| | (iii) Tertiary Nodes: | 7 |
| 4. | Average length of | 3.0 cm |
| | primary internode: | |
| 5. | Leaf apex shape: | MUCRONATE (rounded), CUSPIDATE |
| 6. | Leaf shape: | PALMATELY LOBED, CORDATE |
| 7. | Leaf lamina base shape: | HASTATE |
| 8. | Petiole shape: | LONG, HAIRY |
| 9. | Color of Upper | YELLOW GREEN (147A) |
| | leaf surface: | |
| 10. | Color of lower | YELLOW GREEN (146A) |
| | leaf surface: | |
| 11. | Number of trichomes: | 3.5/mm² |
| 12. | Trichome ratio: | 2:1 |
| | (lower leaf/upper leaf) | |
| 13. | Time for flowering: | FEBRUARY END |
| 14. | Lastingness of bloom: | 3 MONTHS |
| 15. | Flower shape: | TUBULAR |
| 16. | Pedicel length: | 2.5 mm |
| 17. | Pedicel color: | YELLOW GREEN ( ) |
| 18. | Calyx diameter: | 5 mm |
| 19. | Calyx color: | YELLOW GREEN (146A) |
| 20. | Corolla: | FINE, POLY PETALOUS, TWO |
| | | POSTERIOR PETALS, LARGER WITH |
| | | REDDISH PURPLE MARKINGS |
| 21. | Corolla color: | RED PURPLE (74B) |
| 22. | Pubescence of corolla: | ABSENT |
| 23. | Number of anthers: | 10 |
| 24. | Color designation: | YELLOW ( ) |
| 25. | Stigma: | |
| 26. | Ovaries: | ONE SUPERIOR, PENTA |
| | | CARPELLARY SYNCARPOUS OVARY |
| 27. | Color of stigma: | MAGENTA (RED PURPLE) (78B) |
| 28. | Color of ovaries: | WHITE ( ) |

Field Experiments

Out of these 4 progenies obtained preliminary analysis revealed encouraging results for BSP-4, as the plant showed high vigor in biomass production and higher oil yield. So the plant BSP- 4 was taken to the field for further evaluation. To variously compare the BSP-4 accession with the cultivar accessions ‘Bipuli’, ‘Hemanti’ and ‘Kunti’, field plot experiments were carried out over two winter-summer cropping seasons (1998-1999) and (1999-2000) at the experimental farm of Central Institute of Medicinal and Aromatic Plants, at Lucknow India (26.5° N 80.5° E and of 120 m altitude, in subtropical north Indian plains). The soil in the field used was sandy loam in texture, alkaline in reaction (pH, 7.8), low in available N (155 kg/ha) and medium in available phosphorous (30.4 kg P₂O₅/ha) and exchangeable potassium (120 kg K₂O/ha). In October 1998, six glasshouse-grown plants of each accession were transferred to the field trial. The cuttings drawn from each of the 4 accessions were planted in randomized blocks replicated three times, in January 1999. The field blocks used were given a uniform dose of 100 kg/ha P₂O₅ and 60 kg/ha K₂O. Nitrogen was applied in three splits while phosphorus and potassium were applied at the time of planting. A light irrigation was given just after the planting of cuttings and plots were manually weeded and irrigated at regular interval of two weeks throughout the cropping season. The crops were allowed to grow until June when observations on the morphology of the plants were recorded and crops harvested to estimate the yield of herbage as well as yield and quality of essential oil. About 300 g sample of herbage harvested from each plot was distilled to determine its oil content. The oil samples were analyzed by GC, GC-MS procedures. The 4 accessions were similarly grown and assessed in the 1999-2000 winter-summer cropping season at Lucknow. The parameters on which observations were recorded were averaged for the two seasons for presentation in Tables 3 and 4.

TABLE 3
The variation observed in the expression of the essential oil yield
related traits among 4 accessions in the rose scented geranium
Pelargonium graveolens

| | Essential oil yield parameter | | | |
|---|---|---|---|---|
| | Plant | | | Number of |
| | height | Canopy | Herb Yield | branches/ |
| Acc. No. | (cm) | Size (m²) | (kg/plant) | plant |
| Bipuli | 50 ± 2 | 0.81 ± 0.1 | 3.3 ± 0.1 | 25 ± 2 |
| Hemanti | 55 ± 2 | 0.80 ± 0.2 | 3.9 ± 0.5 | 30 ± 4 |
| Kunti | 40 ± 4 | 0.58 ± 0.2 | 2.0 ± 0.1 | 22 ± 1 |
| BSP-4 | 82 ± 6 | 1.42 ± 0.4 | 11.1 ± 0.9 | 47 ± 4 |
| Mean ± (SEM) | 53 ± 5 | 0.73 ± 0.14 | 3.9 ± 1.3 | 27 ± 4 |
| CD at 5% | 5 | 0.30 | 0.6 | 4 |

| | Essential oil yield parameter | | | |
|---|---|---|---|---|
| | | | Leaf | |
| | Number of | | petiole | |
| | leaves/ | Leaf/stem | length | Leaf Area |
| Acc. No. | plant | ratio (cm) | (cm) | (cm²) |
| Bipuli | 1437 ± 100 | 0.7 ± 0.1 | 9.7 ± 1.8 | 70 ± 7 |
| Hemanti | 1801 ± 167 | 0.9 ± 0.1 | 11.1 ± 1.4 | 68 ± 2 |
| Kunti | 1363 ± 177 | 1.2 ± 0.1 | 9.1 ± 1.1 | 108 ± 5 |
| BSP-4 | 4632 ± 201 | 0.9 ± 0.5 | 24.6 ± 2.8 | 101 ± 16 |
| Mean ± (SEM) | 2445 ± 500 | 1.0 ± 0.07 | 11.7 ± 2.3 | 83 ± 6 |
| CD at 5% | 697 | 0.2 | 3.8 | 24 |

| | Essential oil yield parameter | |
|---|---|---|
| | | Oil Yield/ |
| | Oil | plant (g/ |
| Acc. No. | Content % | plant) |
| Bipuli | 0.25 ± 0.1 | 8.2 ± 0.1 |
| Hemanti | 0.20 ± 0.1 | 7.8 ± 0.2 |
| Kunti | 0.3 ± 0.1 | 6.0 ± 0.5 |
| BSP-4 | 0.37 ± 0.1 | 41.0 ± 0.9 |
| Mean ± (SEM) | 0.29 ± 0.2 | 19.3 ± 8.0 |
| CD at 5% | 0.08 | 11.7 |

TABLE 4
The variation in the expression of essential oil quality among 4
accessions, in the rose scented geranium Pelargonium graveolens
% content of terpenoid in essential oil

| Accession | Citronellol | Geraniol | Isomethone | Linalool |
|---|---|---|---|---|
| Bipuli | 34.5 ± 3.4 | 21.8 ± 3.3 | 7.7 ± 0.6 | 4.3 ± 1.6 |
| Hemanti | 50.6 ± 1.4 | 1.2 ± 0.3 | 12.4 ± 0.7 | 1.1 ± 0.1 |
| Kunti | 13.0 ± 1.0 | 43.7 ± 1.4 | 10.5 ± 0.7 | 6.6 ± 0.1 |
| BSP-4 | 30.6 ± 3.0 | 28.7 ± 6.0 | 8.4 ± 0.5 | 4.7 ± 0.1 |
| Means ± | 26.2 ± 5.6 | 27.2 ± 6.2 | 9.5 ± 1.0 | 6.2 ± 1.3 |
| SEM | | | | |
| CD at 5% | 6.5 | 4.3 | 1.9 | 2.7 |

| | Cis rose | Trans rose | | Citronellyl |
|---|---|---|---|---|
| Accession | oxide | oxide | Menthone | formate |
| Bipuli | 0.6 ± 0.3 | 0.3 ± 0.1 | 0.1 ± 0.1 | 7.8 ± 0.3 |
| Hemanti | 0.8 ± 0.1 | 0.5 ± 0.1 | 0.1 ± 0.1 | 13.8 ± 0.8 |
| Kunti | 0.1 ± 0.1 | 0.1 ± 0.1 | 0.2 ± 0.1 | 0.6 ± 0.1 |
| BSP-4 | 0.4 ± 0.1 | 0.2 ± 0.1 | 0.2 ± 0.1 | 6.6 ± 0.5 |
| Means ± | 0.3 ± 0.1 | 0.2 ± 0.1 | 0.2 ± 0.1 | 4.5 ± 0.4 |
| SEM | | | | |
| CD at 5% | 0.3 | 0.1 | 0.1 | 1.0 |

| | Geranyl | 10-epi-Y- | 6,9- | Decanoic |
|---|---|---|---|---|
| Accession | formate | eudesmol | quaiadiene | acid |
| Bipuli | 2.1 ± 0.2 | 5.7 ± 0.3 | 0.1 ± 0.1 | ND^{a} |
| Hemanti | 0.2 ± 0.1 | 2.4 ± 0.2 | 0.4 ± 0.1 | ND |
| Kunti | 0.2 ± 0.1 | 4.1 ± 0.1 | 2.0 ± 0.1 | 2.8 ± 0.1 |
| BSP-4 | 2.9 ± 0.2 | 5.4 ± 0.3 | 0.1 ± 0.1 | 0.1 ± 0.1 |
| Means ± | 0.9 ± 0.4 | 3.7 ± 0.8 | 1.8 ± 0.7 | 1.5 ± 0.7 |
| SEM | | | | |
| CD at 5% | 0.5 | 0.5 | 0.1 | 0.1 |

| | | Phenyl ethyl |
|---|---|---|
| Accession | Isodecanoic | tiglate |
| Bipuli | ND | 0.8 ± 0.3 |
| Hemanti | ND | 0.8 ± 0.2 |
| Kunti | 0.6 ± 0.2 | 0.9 ± 0.1 |
| BSP-4 | ND | 0.8 ± 0.1 |
| Means ± | 0.4 ± 0.2 | 1.0 ± 0.6 |
| SEM | | |
| CD at 5% | 0.1 | 0.5 |

^{a}ND = not detected

GC and GC-MS Analysis

GC analysis of the essential oils was performed on a Perkin-Elmer gas chromatograph 8500 equipped with FID, using two fused silica capillary columns BP-1 coated with dimethyl siloxane (30 m×0.25 mm×0.25 μm film thickness) and BP-20 coated with carbowax 20M (20 m×0.25 mm×0.25 μm thickness), carrier gas nitrogen at 10 psi inlet pressure and temperature programmed to 60-220° C. at 5° C./min. For BP-20 column and split ratio of 1:80. GC-MS was performed on Schimadzu QP-2000 instrument using ULBON. HR-1 fused silica column (50 m×0.25 mm×0.25 μm film thickness), temperature programmed to 100°-250° C. at 10° C./min, carrier gas helium at 2 ml/min, MS conditions of El mode 70 eV and ion source temperature of 250° C.

Identification of the Compounds

Compounds were identified by comparing the retention indices (relative to C8-C21 alkanes) with those reported in literature by peak enrichment on coinjection with standards wherever possible and by comparison of mass spectra of the peak with those of compounds reported in literature (Jennings, W. & T. Shibamoto, 1980. Qualitative analysis of flavour and fragrance volatile by capillary GC, Academic Press Inc., New York.; Adams, R. P., 1990. Identification of essential oils by ion trap mass spectroscopy. Academic Press, San Diego, Calif.). Relative amounts of individual components were estimated based on peak areas on BP-1 column without FID response correction.

Molecular Analysis of Hybrids

DNA was isolated from young leaves (1 g) taken from mature plants following the reported protocol (Khanuja, S. P. S., Shasany, A. K., Darokar, M. P., and Kumar, S., 1999, Rapid isolation of PCR amplifible DNA from dry and fresh samples of plant producing large amounts of secondary metabolites and essential oil by modified CTAB procedure. Plant Molecular Biology, 17:74.) and was digested with EcoRI restriction endonucleases. A set of twenty decanucleotide primers (M/S Bangalore Genie, India) were used for PCR amplification. Polymerase chain reaction (PCR) was carried out in 25 μl reaction volume, containing 20-40 ng of plant genomic DNA, 125 μlM Of MgCl₂ buffer, 100, μM of each dNTP, 5 p motes of primer and 0.2 units of Taq DNA polymerase. Amplification was carried out in DNA Engine PTC 200 (M J Research, USA) thermal cycler programmed for 45 cycles of 1 min at 94° C., I at 36° C. and 2 min at 72° C. The amplification cycle was concluded with final extension at 72° C. for 5 min. Amplification products were electrophoresed in 1.2% (w/v) agarose gel, visualized by ethidium bromide (0.5 μgml⁻¹) staining. The pictures of the gel were scanned for the presence of polymorphic fragments which were scored for the presence (+) or absence (−) of bands. The data so generated was used for calculating the index of genetic similarity using Nei and Li's matching co-efficient method (Nei, M. and Li, W. H., 1979. Mathematical model for studying genetic variation in terms of restriction endonuclease. Proc. Natl. Acad. Sci. U.S.A.,74 : 5267-5273.). The similarity indices were calculated by using the formula: Number of similar bands between the two accessions/total number of bands in the two accessions×2 and present in Table 5.

TABLE 5
Average similarity indices of BSP-4 with the cultivar accessions
‘Bipuli’, ‘Hemanti’ and ‘Kunti’ in rose
scented geranium Pelargonium graveolens

| Accession | Bipuli | Hemanti | Kunti | BSP-4 |
|---|---|---|---|---|
| Bipuli | 1.00 | | | |
| Hemanti | 0.81 | 1.00 | | |
| Kunti | 0.83 | 0.78 | 1.00 | |
| BSP-4 | 0.76 | 0.75 | 0.67 | 1.00 |

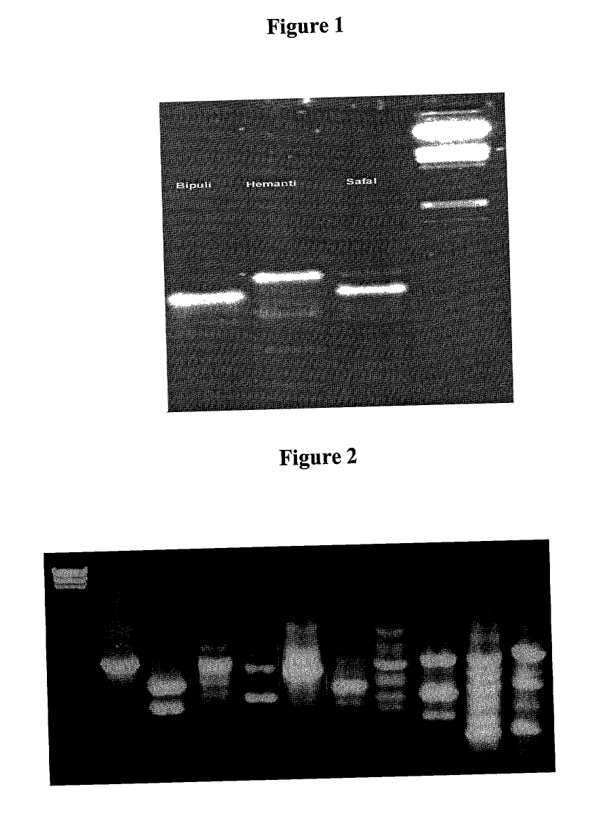

The similarity indices arrived at using 20 decanucleotide primers (MAP01 to MAP20: AAATCGGAGC, GTCCTACTCG, GTCCTTAGCG, TGCGCGATCG, AACGTACGCG, GCACGCCGGA, CACCCTGCGC, CTATCGCCGC, CGGGATCCGC, GCGAATTCCG, CCCTGCAGGC, CCAAGCTTGC, GTGCAATGAG, AGGATACGTG, AAGATAGCGG, GGATCTGAAC, TTGTCTCAGG, CATCCCGAAC, GGACTCCACG, AGC;CTGACGC, respectively) in the RAPD analysis are given in the Table 4. Table 4 indicated that the accession BSP-4 was more similar to the parent varieties ‘Bipuli’ and ‘Hemanti’ (more than 75%) than ‘Kunti’ (67%). Further the RAPD profile of ‘Safal’ showed co-dominance inheritance from ‘Bipuli’ and ‘Hemanti’ (FIG. 1).

Distinctiveness of BSP-4

The accession ‘BSP-4’ expressed most of the characters at much higher levels than the corresponding measurements in the rest of the accessions. In ‘BSP-4’, the leaf/stem ratio and leaf area measurements fell within the ranges covered for these characters by the cultivar accessions. As compared to ‘Bipuli’, ‘BSP-4’ was 1.6 fold taller in height, had 1.7 fold larger canopy, yielded 3.5 fold more herbage, had 2 fold more branches, 3 fold more leaves, 3.5 fold longer leaf petiole, 1.4 fold more oil content in herbage and gave 5 fold more oil yield (Table 2). Apparently the accession ‘BSP-4’ demonstrated the kind of vigour associated with hybrids or transgressive segregants. The variation observed for the essential oil quality parameters among the seven accessions in terms of the 14 terpenoid components that could be identified are summarized in the Table 3. The oil of the accession BSP-4 had geraniol to citronellol proportion as 1:1 and had other parameters widely different from those observed for the oils of the other accessions. The cis and trans rose oxides and citronellyl fonnate contents in the oil of ‘BSP-4’ accession were lower than in the oils of the accessions ‘Bipuli’ and ‘Hemanti’. The essential oil of the accession ‘BSP-4’ had low concentrations of 6,9-guaiadiene, decanoic acid and, isodecanoic acid, like in the oils of the accessions ‘Bipuli’ and ‘Hemanti’. These observations described above have demonstrated that the accession ‘BSP-4’ of P. graveolens differed from the cultivar accessions ‘Bipuli’, ‘Kunti’ and ‘Hemanti’ not only in some of the morphological and essential oil characteristics but also in their DNA profiles.

The accession ‘BSP-4’ demonstrated the expression of essential oil yield related characters at much higher levels than by all the accessions studied. The terpenoid profile of the essential oil of the accession ‘BSP-4’ also appeared to be unique, in that it had equally high concentrations of citronellol and geraniol and very high concentration of total rhodinols. Considering all the essential oil yield related characters together with hierarchical relationships arrived at by DNA fingerprinting, it is possible to surmise that the accession ‘BSP-4’ may be a hybrid between the accession ‘Bipuli’ and ‘Hemanti’. The accession ‘BSP-4’ is novel, unique and has highly useful combination of yield, essential oil quality related characteristics, and this can be used for commercial cultivation to extract high value essential oil having utility for industrial and pharmaceutical purposes. Finally, the new plant was selected for its high quality essential oil and the genotype can be used in the future for plant improvement. The accession BSP-4 is the plant of this invention and was named as ‘Safal’.

Randomly Amplified Polymorphic DNA Analysis

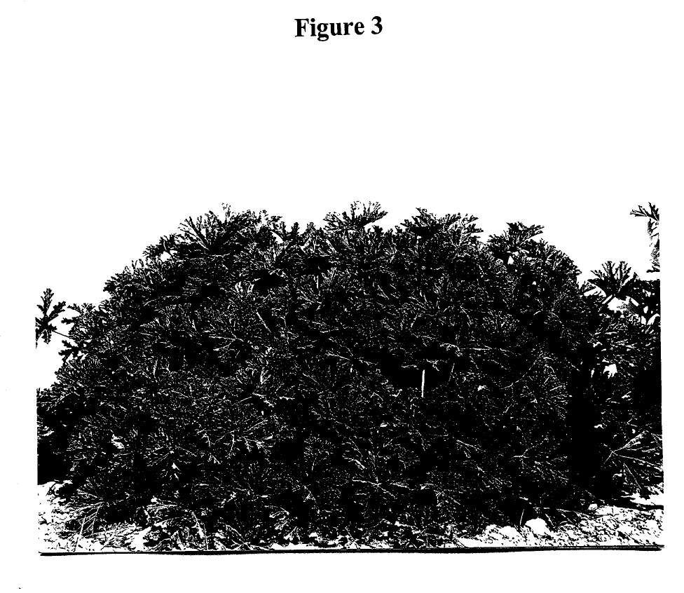

The RAPD profiles of the plant ‘Safal’ were unambiguously able to establish its distinct identity as completely different from the parent plant ‘Bipuli’ as well as the known varieties ‘Kunti’ and ‘Hemanti’. The plant of the present invention was developed by screening the ‘Bipuli’ seed progenies and differentiated as distinct, unique and novel at DNA level. The plant is having desirable morphological and economical traits in a rare unmatchable combination and is available only with us in CIMAP. The primers with the sequence AAATCGGAGC, GTCCTACTCG, TGCGCGATCG, AACGTACGCG, CGGGATCCGC, GCGAATTCCG, CCCTGCAGGC, CCAAGCTTGC, AAGATAGCGG, GGATCTGAAC, were used to develop a unique and distinct PAPD profile of the Plant (FIG. 2). The whole plant ‘Safal’ has been shown in FIG. 3 wherein the canopy and the shape of the leaves are quite apparent.

1. A new and distinct plant of Pelargonium graveolens ‘Safal’, developed through progeny screening from the seeds obtained from the plant cultivar ‘Bipuli’ of Pelargonium graveolens, possessing the following combination of characters:
a. the plant is a hybrid between cultivar ‘Bipuli’ and cultivar ‘Hemanti’ as the pollen donor as indicated by the co-dominance RAPD pattern obtained by the random primer 5′AACGTACGCG3′.
b. the said plant possesses the following botanical description
Stem shape: ROUNDED
Stem habit: RAMIFIED
Number of nodes
(iv) Primary Nodes: 4
(v) Secondary Nodes: 25 to 30
(vi) Tertiary Nodes: 7
Average length of primary internode: 3.0 cm
Leaf apex shape: MUCRONATE (rounded), CUSPIDATE
Leaf shape: PALMATELY LOBED, CORDATE
Leaf lamina base shape: HASTATE
Petiole shape: LONG, HAIRY
Color of upper leaf surface: YELLOW GREEN (147A)
Color of lower leaf surface: YELLOW GREEN (146A)
Number of trichomes: 3.5/mm²
Trichome ratio: 2:1 (lower leaf/upper leaf)
Time for flowering: FEBRUARY END
Lastingness of bloom: 3 MONTHS
Flower shape: TUBULAR
Pedicel length: 2.5 mm
Pedicel color: YELLOW GREEN
Calyx diameter: 5 mm
Calyx color: YELLOW GREEN (146A)
Corolla: FINE, POLY PETALOUS, TWO POSTERIOR PETALS LARGER WITH REDDISH PURPLE MARKINGS
Corolla color: RED PURPLE (74 B)
Pubescence of corolla: ABSENT
Number of anthers: 10
Color designation: YELLOW
Stigma:
Ovaries: ONE SUPERIOR, PENTA CARPELLARY SYNCARPOUS OVARY
Color of stigma: MAGENTA (RED PURPLE) (78B)
Color of ovaries: WHITE
c. the said plant possesses vigorous and rapid vegetative growth with higher plant height of up to 82±6 cm, higher canopy size of up to 1.42±0.4 m², higher herb yield of up to 11.1±0.9 kg per plant, higher leaf area of up to 101±16 cm², higher oil content up to 0.35 to 0.40% and higher oil yield of up to 41 g/plant,
d. the said plant produces an essential oil with the following composition Citronellol 30.6±3.0, Geraniol 28.7±6.0, Isomenthone 8.4±0.5, Linalool 4.7±0.1, Cis rose oxide 0.4±0.1, trans rose oxide 0.2±0.1, Menthone 0.2±0.1, Citronellyl formate 6.6±0.5, Geranyl formate 2.9±0.2, 10-epi-γ-eudesmol 5.4±0.3, 6,9-guaiadiene 0.1±0.1, Decanoic acid 0.1±0.1, Phenyl ethyl tiglate 0.8±0.1, which may not be construed to be limited to these values,
e. the said plant has distinct molecular profile by random amplified polymorphic DNA (RAPD) using 10 random primers (AAATCGGAGC, GTCCTACTCG, TGCGCGATCG, AACGTACGCG, CGGGATCCGC, GCGAATTCCG, CCCTGCAGGC, CCAAGCTTGC, AAGATAGCGG, GGATCTGAAC) distinguishing the plant from the other existing varieties, and
f. the said plant is able to produce highest herbage, oil yield per plant as compared to any other existing varieties.
